(19) United States
(12) Patent Application Publication
(10) Pub. No.: US 2007/0119984 A1
(43) Pub. Date: May 31, 2007

(54) Pressure chamber nozzle assembly

(76) Inventor: John R. Woods, Woodland Hills, CA (US)

Correspondence Address: PILLSBURY WINTHROP SHAW PITTMAN LLP, P.O BOX 10500, McLean, VA, 22102, US

(21) Appl. No.: 11/698,528

(22) Filed: Jan. 26, 2007

Related U.S. Application Data
(63) Continuation-in-part of application No. 10/831,913, filed on Apr. 26, 2004.

(51) Int. Cl. B05B 7/32; F23D 14/28; A61M 11/06; B65D 83/00

(52) U.S. Cl. 239/337; 239/338; 222/402.2

(57) ABSTRACT

A spray nozzle assembly is disclosed which provides for increased atomization, a finer spray, and a reduced tendency of the apparatus to clog. The nozzle assembly includes a discharge opening, an expansible bladder, and optionally, a pressure chamber through which the spray material must pass prior to exiting the spray container through the discharge opening. As material exiting the container passes through the expansible bladder and the pressure chamber, pressure is built up, which results in increased shearing and atomization of the spray material.

### CROSS-REFERENCE TO RELATED APPLICATIONS

This application is a continuation-in-part of pending U.S. patent application Ser. No. 10/831,913, filed Apr. 26, 2004.

### BACKGROUND OF THE INVENTION

1. Field of the Invention

This invention generally relates to a nozzle assembly for use with a variety of spray apparatuses, including for example, a spray can. More particularly, the invention relates to a nozzle assembly configured to induce pressure build-up so that the substance being sprayed is atomized to a higher degree, with a variable orifice or discharge opening. The invention includes a wire wound around the nozzle orifice to adjust the orifice size. The wire further provides a manner of restriction that contributes to additional pressure build-up for higher atomization.

2. Description of Related Art

The practice of dispensing sprayable materials through traditional aerosol spray can valve assemblies has presented problems in that the nozzle on occasion may clog, particularly when the spray can is used infrequently. Additionally, in some instances, a greater degree of atomization may be desired for optimum functioning of the spray device. Furthermore, the practice of dispensing heavy and particulate materials through traditional aerosol spray can valve assemblies in the aerosol industry has presented problems in that the heavy and particulate materials to be dispersed have a tendency to clog up the valve assemblies. These heavy and particulate materials may include exterior stucco, heavy sand finishes, drywall and acoustic ceiling patching materials, fire suppressant materials, adhesive and bonding materials, and even culinary sauces.

As is well known in the art, traditional aerosol spray cans may be filled with material for dispensing, such as paints. Similarly, a traditional aerosol spray can may be filled with heavy and particulate materials for spraying.

However, because of the placement of the spray nozzle assembly in traditional aerosol spray cans, both traditional spray materials as well as the heavy and particulate materials will clog up the nozzle or valve assemblies and render the aerosol spray cans inoperative. For example, constant operation of these aerosol spray cans in spraying heavy and particulate materials is not possible due to the inconsistent ability of these traditional nozzle or valve assemblies to dispense these materials without clogging.

U.S. Pat. No. 5,715,975, issued to Stern et al., discloses an aerosol spray texturing device that is comprised of a container, a nozzle, a valve assembly, and an outlet. The valve assembly in the '975 patent is located in the upper section of the container near the nozzle. Although the nozzle tube of the device in the '975 patent may be configured to spray texture materials, the device in the '975 patent still has the problem of clogging or packing of the valve assembly by the particulates contained in the texture material for spraying, especially if the particulates are large, like those found in stucco or other heavy and particulate materials mentioned above.

U.S. Pat. No. 5,645,198, also to Stern, discloses a number of different ways in which texture material may be dispensed from a spray can to achieve a variety of different textures. The general concept is that such different textures may be achieved by varying the diameter of the outlet orifice. Such variation in diameter of the outlet orifice may be achieved, for example, (a) by using a plurality of different straws, each-having a different internal diameter, (b) through use of a rotatable cap having a plurality of differently sized holes for outlet orifices, (c) through use of a deformable straw with a constricting sleeves or (d) through use of a deformable outlet passageway with a deformable rotating cap. Such variety in textures which being available from one can is highly desirable in the eye of the consumer.

Therefore, a long-standing need has existed to provide an apparatus that may be used to readily apply spray materials, including heavy and particulate materials, in aerosol form with increased atomization and without clogging of the nozzle. In some instances, it may further be desirable to spray such materials in more than one texture. Furthermore, such spray should be contained in a hand-held applicator so that the materials may be conveniently stored, as well as dispensed in a simple and convenient manner without clogging or packing the spray nozzle assembly of the applicator. Lastly, there is also a need to optimize the pressure that can be built up by the spray nozzle assembly to achieve the optimal level of atomization and shearing to the sprayable material.

### BRIEF SUMMARY OF THE INVENTION

The present invention provides a spray nozzle assembly for use in spray apparatuses, such as an aerosol spray can, which is configured to spray material with an increased pressure, an increased degree of atomization and reduced clogging over traditional nozzle or valve assemblies known in the art. Such improved functionality stems from the inclusion of a pressure chamber in the dispensing assembly prior to the discharge opening. The nozzle assembly is further capable of spraying a wide variety of different types of materials and create a wide variety of spray textures by adjusting the nozzle orifice size. The sprayable materials include, but are not limited to, paints, resins, other liquids and viscous materials or materials with large particulates.

The nozzle assembly according to the present invention uses many of the same elements as conventional nozzle assemblies, but incorporates a pressure chamber formed in the exit passageway so that material exiting the container passes through the pressure chamber prior to exiting the system through the discharge opening or orifice. The inclusion of the pressure chamber as part of the exit passageway allows for pressure build-up prior to the spray material's exit of the dispensing system and generates increased shearing and atomization. The increased pressure also leads to a reduced tendency for the nozzle to clog.

In embodiments, there is provided a spray nozzle assembly comprising a dip tube with a top opening and a bottom opening, wherein the bottom opening is configured to be in flowable communication with a sprayable material, an actuator coupled to the top end of the dip tube, an expansible bladder located at the top opening of the dip tube and configured to be in flowable communication with the top opening when the actuator is depressed, wherein the expansible bladder expands in diameter from a resting diameter when the sprayable material passes through, and a discharge opening in flowable communication with the expansible bladder, wherein sprayable material is dispensed through the discharge opening when the actuator is depressed. The resting diameter is diameter of the bladder when no sprayable material is passing through to generate pressure in the bladder. In further embodiments, the spray nozzle of claim further includes a pressure chamber located in between the expansible bladder and the discharge opening and establishing flowable communication from the expansible bladder to the discharge opening, wherein the discharge opening has a diameter that is smaller than a diameter of a portion of the expansible bladder that immediately abuts the discharge opening.

In particular embodiments, a system for using the spray nozzle assembly may comprise a container, a sprayable material being held in the container, and the spray nozzle assembly described above being attached to the container and adapted to dispense the sprayable material. The dip tube of the nozzle assembly is at least primarily disposed inside the container.

The present embodiments also provide a method of applying a sprayable material onto a surface area, the method comprising storing a sprayable material in a dispensing container having the above-described spray nozzle assembly for application to a surface area, and selectively applying the sprayable material onto the surface area such that a layer of the sprayable material is formed.

Embodiments of the invention subject the spray material to increased pressure prior to dispensing through the discharge opening because the flow path that the sprayable material travels through subjects the sprayable material to pressure build up. The sprayable material first passes through the expansible bladder and subsequently through the pressure chamber. In both instances, high pressure is generated by the sprayable material which contributes to greater shearing and atomization of the sprayable material prior to exiting from the discharge opening. Thus, the pressure chamber in combination with the expansible bladder facilitates much greater compression of the sprayable material prior to exiting than previously known nozzle assemblies. The increased pressure also leads to a reduced tendency for the nozzle to clog.

The nozzle assembly, expansible bladder and pressure chamber of the above embodiments may be used with any conventional aerosol or spray container or system. For example, the nozzle assembly and pressure chamber may be used with a variety of spray devices like a spray gun hopper. As with conventional aerosol or spray containers or systems, the actuator allows the user to selectively open or close the spray nozzle assembly so that the sprayable material is dispensed when desired.

### BRIEF DESCRIPTION OF THE DRAWINGS

For a better understanding of the present embodiments, reference may be had to the accompanying figures.

FIG. 1 is a front view of a pressure chamber valve assembly in accordance with one embodiment of the present invention;

FIG. 2 is a cross-sectional view of a pressure chamber valve assembly in accordance with the embodiment of the present invention shown in FIG. 1, taken along the “A-A” line of FIG. 1;

FIG. 3 is a side view of a pressure chamber valve assembly in accordance with one embodiment of the present invention;

FIG. 4 is a perspective view of a spray device which incorporates the pressure chamber valve assembly in accordance with one embodiment of the present invention;

FIG. 5 is a cross-sectional view of a pressure chamber valve assembly in accordance with one embodiment of the present invention;

FIG. 6 is a cross-sectional view of a pressure chamber valve assembly in accordance with one embodiment of the present invention;

FIG. 7 is a cross-sectional view of a pressure chamber valve assembly in accordance with one embodiment of the present invention;

FIG. 8 is a cross-sectional view of a spray nozzle assembly in accordance with one embodiment of the present invention; and

FIG. 9 is a cross-sectional view of a system having a spray nozzle assembly in accordance with one embodiment of the present invention.

### DETAILED DESCRIPTION

The present invention provides a valve assembly for use in an aerosol spray can which is configured to spray material with an increased pressure, an increased degree of atomization and reduced clogging over traditional valve assemblies known in the art. Such improved functionality stems from the inclusion of a pressure chamber in the dispensing assembly prior to the discharge opening. The nozzle assembly is capable of spraying a wide variety of different types of materials. Such materials include, but are not limited to, paints, resins, other liquids and viscous materials or materials with large particulates. The present invention may also be used in a wide variety of spray devices, including but not limited to, spray guns, spray hoppers, aerosol cans and canisters, and the like.

The present invention provides an inexpensive and economical means for dispensing materials with an increased degree of atomization and a reduced incidence of clogging of the nozzle. Such reduced clogging is believed to be facilitated by the increased spray pressure and resulting atomization of the material which is being sprayed. Not only is the present invention easy to manufacture and assemble, but the reduced incidence of clogging results in increased user satisfaction and is expected to lead to a lower incidence of returns due to clogged nozzles.

When the present invention is used in association with known texture-modifying structures, it also provides an inexpensive and economical means for matching surface texture of a repaired or patched texture surface area. Since the spray-on hardenable texture material covers the repaired or patched area and visually assumes the surface texture of the surrounding patched or repaired surface, this results in the user seeing an improvement in the appearance of patched or repaired areas on a textured surface.

Aerosol assemblies are well known in the art. Generally, they comprise a container, a valve assembly, and an actuator member. As is also well known in the art, depressing the actuator member moves the valve assembly into its open position in which an exit passageway is defined from the interior of the container to the exterior of the container. When in the open position, the pressure chamber, dip tube and discharge opening are configured to be in flowable communication so that sprayable material in the container can be dispensed. The exit passageway generally terminates in a discharge opening formed in the actuator member.

The nozzle assembly according to the present invention uses many of the same elements as prior art nozzle assemblies, but additionally includes a pressure chamber in the exit passageway so that material exiting the container passes through the pressure chamber prior to exiting the system through the discharge orifice or opening. The inclusion of the pressure chamber as part of the exit passageway allows for pressure build-up prior to the spray material's exit of the dispensing system.

The invention subjects the spray material to increased pressure prior to dispensing. This assists in shearing the material and provides increased atomization of the spray material. The increased pressure also leads to a reduced tendency for the nozzle to clog. Preventing clogging is important, especially for acoustic materials used for creating irregular surface textures. These materials are useful for repairing and matching existing surfaces, such as for example, stucco walls. Acoustic materials can cause clogging due to the particulates that they contain in order to form a layer having the irregular surface texture. For example, acoustic materials generally contain particulate filler materials, such as for example, calcium carbonate, silica, talc, wollastonite, and the like. The particulate filler material desirably has various particle sizes and shapes so that when the acoustic material is applied onto the desired surface, the particulate forms irregular surface textures.

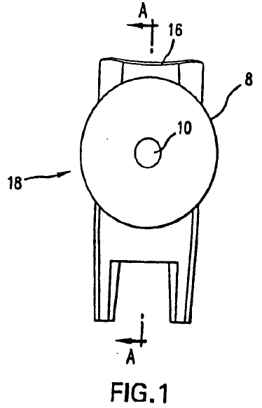

FIG. 1 is a front view of a valve assembly 18 in accordance with one embodiment of the present invention. This figure shows the variable nozzle 8 having a variable discharge opening 10 which is aligned with the pressure chamber 14 (not shown) discharge opening or exit orifice, and the actuator 16. In some embodiments, the variable nozzle 8 is coupled to the valve assembly by screwing threads. The user may tighten or loosen the variable nozzle 8 to enlarge or reduce the size of the variable discharge opening 10. That is, when the variable nozzle 8 is tightened, the rubber is pushed back and the variable discharge opening 10 is able to discharge more spray texture material, with less fine particles. In contrast, when the variable nozzle 8 is loosened, the rubber is relaxed, and the variable discharge opening 10 discharges less spray material, with finer particles. Thus, a smaller diameter variable discharge opening 10 results in a more spray texture, while a larger diameter variable discharge opening results in a courser spray texture.

The variable nozzle 8 is one of many features of the present embodiments which may be added to permit the user to vary the resulting texture of the spray material being dispensed. Furthermore, such texture-varying means are not required to use the valve assembly according to the present embodiments.

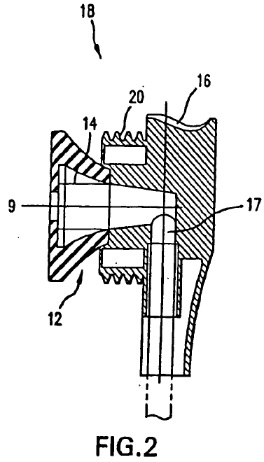

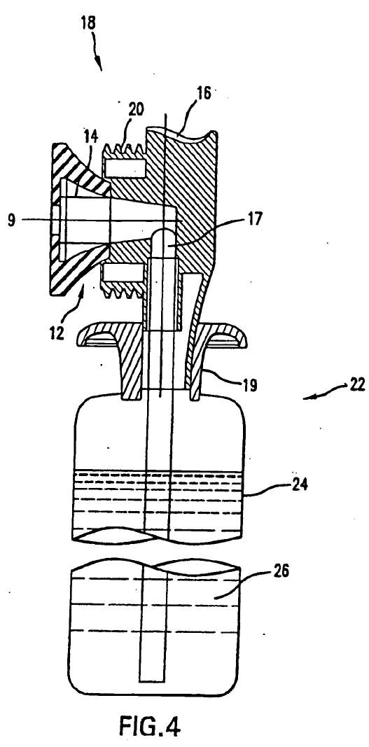

FIG. 2 is a cross-sectional view of a valve assembly 18 in accordance with the embodiment of the present invention shown in FIG. 1, taken along the “A-A” line of FIG. 1. As shown, this figure does not include a variable spray nozzle. The pressure chamber 14 is generally flared, with the flare starting back where the pressure chamber 14 is coupled to the vertical portion 17 of the discharge passageway. The vertical portion 17 of the discharge passageway comprises, at a minimum, a dip tube. The dip tube may be of sufficient length so as to extend into container 24 (See FIG. 4) and only along a portion of its height. Alternatively, the dip tube may extend to the bottom of the container 24.

As used herein, the term “discharge passageway” will refer to that structure or those structures through which the spray material passes en route from the holding container (not shown), through the pressure chamber discharge opening 9, to the variable discharge opening 10.

In different embodiments, the pressure chamber may take a variety of different shapes. By way of example, and not of limitation, it may be cone-shaped or flare at a greater or lesser angle, it may be bulbous or it may be square or rectangular. Alternatively, the diameter of the discharge passageway may remain the same after it assumes a generally horizontal configuration, and may widen into the pressure chamber at some point subsequent to its turn to the horizontal configuration (that is, from a generally vertical to generally a horizontal configuration).

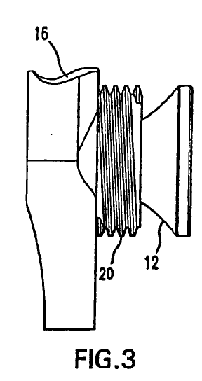

FIG. 3 is a side view of a valve assembly 18 in accordance with one embodiment of the present invention. This figure shows the exterior 12 of the pressure chamber 14, the actuator 16, and threads 20. As will be realized by one of ordinary skill in the art, such threads are simply one means through which a protective cap may be coupled to the present invention.

The valve assembly 18 is preferably generally formed from plastics using means readily known in the art. However, other materials may also be used to form the valve assembly 18, or portions thereof, including, but not limited to, resins or metals. Of course, if the valve assembly 18 is used in association with a variable nozzle 8, the variable discharge opening 10 will preferably be formed from rubber or other readily malleable material.

FIG. 4 is a perspective view of a spray device 22 which incorporates the pressure chamber valve assembly 18 in accordance with one embodiment of the present invention. As may be seen, the spray device 22 generally includes a valve assembly 18, a bushing 19, a container 24 and spray material 26.

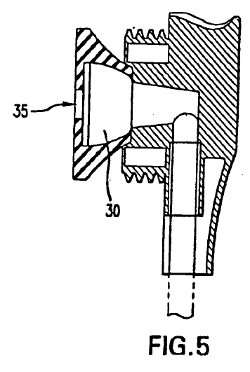

FIG. 5 is a cross-sectional view of another embodiment of the present invention. As may be seen, the pressure chamber 30 depicted is generally bulbous in shape. Adjacent to the pressure chamber 30 is a discharge opening 35 that is adapted to be in flowable communication with the pressure chamber 30. The discharge opening 35 has a diameter that is smaller than a diameter of the portion of the pressure chamber that abuts the discharge opening.

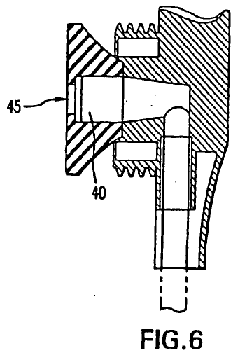

FIG. 6 is a cross-sectional view of another embodiment of the present invention. As may be seen, the pressure chamber 40 depicted is generally square in shape. Adjacent to the pressure chamber 40 is a discharge opening 45 that is adapted to be in flowable communication with the pressure chamber 40. The discharge opening 45 has a diameter that is smaller than a diameter of the portion of the pressure chamber that abuts the discharge opening.

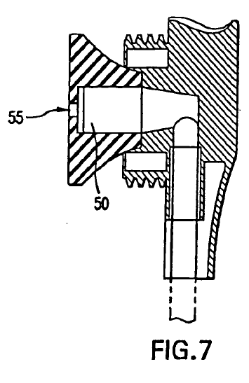

FIG. 7 is a cross-sectional view of another embodiment of the present invention. As may be seen, the pressure chamber 50 depicted is generally rectangular in shape. Adjacent to the pressure chamber 50 is a discharge opening 55 that is adapted to be in flowable communication with the pressure chamber 50. The discharge opening 55 has a diameter that is smaller than a diameter of the portion of the pressure chamber that abuts the discharge opening.

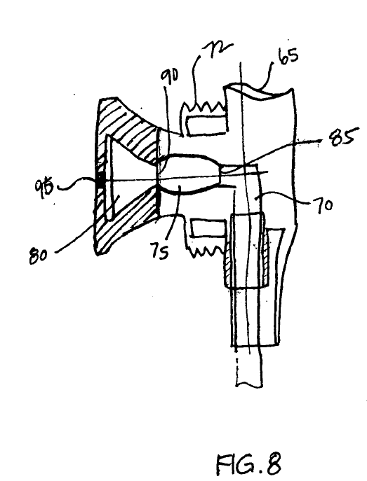

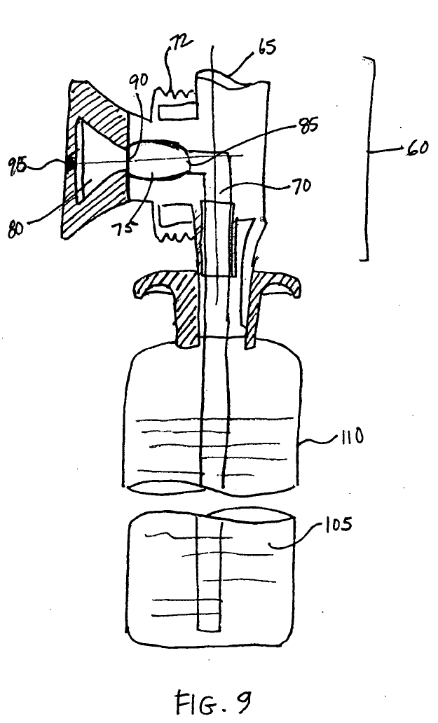

In further embodiments, shown in FIGS. 8 and 9, the spray nozzle assembly 60 may comprise an actuator 65 with a dip tube 70 with a top opening and a bottom opening. The bottom opening is configured to be in flowable communication with a sprayable material, and the top end of the dip tube is coupled to an actuator 65. Threads 72 may be included as means through which a protective cap or other attachments may be coupled to the spray nozzle assembly 60. An expansible bladder 75 is located at the top opening of the dip tube and configured to be in flowable communication with the top opening of the dip tube 70 when the actuator 65 is depressed. Upon depression of the actuator 65, the expansible bladder 75 becomes in flowable communication with the top opening and expands in diameter from a resting diameter when the sprayable material passes through. The resting diameter is the diameter of the expansible bladder 75 when the actuator 65 is not depressed and the bladder 75 is not expanded, e.g., no sprayable material is passing through the bladder.

The expansible bladder 75 comprises a resilient and elastic material, such as for example, natural and synthetic rubber, silicone, and mixtures thereof. Types of rubber may include, but are not limited to, acrylic rubber, butadiene rubber, butyl rubber, chlorobutyl, chlorinated polyethylene, chlorosulphonated polyethylene, epichlorhydrin, ethylene acrylic, ethylene propylene rubber, fluoroelastomers, hyrodogenated nitrile rubber, isoprene rubber, natural rubber, nitrile rubber, perfluoro elastomers, polychloroprene, polynorbornene rubber, polysulphide rubber, polyurethane rubber, silicone rubber, fluorosilicone rubber, styrene butadiene rubber, and tetra-fluoroethylene/propylene. Resilient materials allow the bladder to readily return to its resting size and shape. Thus, a bladder comprising such materials in the aerosol system generates additional shearing and dispersion forces of the dispensed sprayable material through the added pressure created by the bladder in seeking to return to its resting size and shape. The bladder 75 is located at the top opening of the dip tube 70 so that the added pressure can be generated prior to exiting the aerosol assembly. In this manner, the greatest pressure is generated right before exiting the assembly to impart high atomization of the sprayable material.

In other embodiments, the expansible bladder 75 and a pressure chamber 80 may be positioned adjacent to one another and are configured to be in flowable communication with the top opening. As the bladder 75 is located at the top opening of the dip tube 70, the added pressure can be generated and transferred directly into the pressure chamber 80 prior to exiting the aerosol assembly. In this manner, the greatest pressure is generated right before exiting the assembly such that high atomization of the sprayable material is achieved. The bladder 75 has an entry opening 85 into the expansible bladder 75 and an exit opening 90 out of the expansible bladder 75, and these openings each have a diameter smaller than the resting diameter of the expansible bladder 75. The elasticity of the bladder 75 allows it to expand and let in additional sprayable material which generates pressure build up in the bladder 75, as the exit opening 90 is much smaller than the diameter of the expansible bladder 75. The exit opening 90 may be rigid and not expandable so that high pressure is generated from the backflow of the additional sprayable material pushing to exit the smaller exit opening 90. In embodiments, the entry opening 85 of the bladder 75 is larger in diameter than that of the exit opening 90, although not greater than the greatest diameter that the expansible bladder 75 can expand to. In some embodiments, the expansible bladder 75 is adapted to expand up to five times the resting diameter. In other embodiments, the bladder 75 can be expanded to an even greater size depending on the size of the spray nozzle assembly 60.

The atomized and sheared sprayable material subsequently exits the expansible bladder 75 and exits through the discharge opening. In embodiments, the atomized and sheared sprayable material subsequently exits the expansible bladder 75 and enters the pressure chamber 80, which may be configured to be in flowable communication with the expansible bladder 75 when the actuator 65 is depressed. The sprayable material is further atomized and sheared by the increased pressure generated in the pressure chamber 80. In such embodiments, a discharge opening 95 in flowable communication with the pressure chamber 80 has a diameter that is smaller than a diameter of a portion of the pressure chamber 80 that is directly next to or immediately abuts the discharge opening 95, and the material is dispensed through the discharge opening 95 when the actuator 65 is depressed. As a result, the pressure build up is generated as the sprayable material is being dispensed through a smaller discharge opening. In further embodiments, the exit opening 90 of the expansible bladder 75 is greater than the discharge opening 95. In embodiments, the discharge opening 95 has a diameter that is at least three times smaller than the diameter of the portion of the pressure chamber 80 that immediately abuts the discharge opening 95. It is observed that the greater the difference between diameter sizes of the discharge opening 95 and the pressure chamber 80, the greater the pressure is generated which leads to a desirable high shearing and atomization of the sprayable material. Thus, even more pressure is built up in the pressure chamber 80, adding to the amplified compression and further shearing of the material.

Embodiments of the invention subject the spray material to increased pressure prior to dispensing. The present embodiments of the expansible bladder in combination with the pressure chamber facilitates more compression of the sprayable material than previously known nozzle assemblies. This higher level of compression causes better shearing of the material so that the material is sprayed with much higher atomization. The increased pressure also leads to a reduced tendency for the nozzle to clog.

In further embodiments, the pressure chamber 80 flares to a diameter that is larger than the diameter of the dip tube 70. In different embodiments, the pressure chamber 80 can be of various shapes, such as for example, bulbous, flared, square, rectangular, and the like. The shapes are selected to give a great volume size.

As seen in FIG. 9, the spray nozzle assembly 60 described above can be used in an aerosol system 100 for applying a sprayable material 105. The system 100 comprises, in embodiments, a container 110, a sprayable material 105 being held in the container 110, and a spray nozzle assembly 60 attached to the container 110 that sprays the sprayable material 105 from the container 110. As discussed above, the spray nozzle assembly 60 comprises a dip tube 70 with a top opening and a bottom opening, wherein the bottom opening is configured to be in flowable communication with the sprayable material 105, an actuator 65 coupled to the top end of the dip tube 70, an expansible bladder 75 configured to be in flowable communication with the top opening when the actuator 65 is depressed, wherein the expansible bladder 75 expands in diameter from a resting diameter when the sprayable material 105 passes through, and a discharge opening 95 in flowable communication with the expansible bladder 75. In embodiments, a pressure chamber 80 is located between the expansible bladder 75 and the discharge opening 95, and establishes flowable communication from the expansible bladder 75 to the discharge opening 95 when the actuator 65 is depressed. In such embodiments, the discharge opening 95 has a diameter that is smaller than a diameter of a portion of the pressure chamber 80 that immediately abuts the discharge opening 95, wherein sprayable material 105 is dispensed through the discharge opening 95 when the actuator 65 is depressed.

Further embodiments provide a method of applying a sprayable material onto a surface area using the spray nozzle assembly and the aerosol system shown in FIGS. 8 and 9. The method may comprise storing a sprayable material in a dispensing container having the spray nozzle assembly, discussed above, for application to a surface area, and selectively applying the sprayable material onto the surface area such that a layer of the sprayable material is formed.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

What is claimed is:

1. A spray nozzle assembly comprising:
a dip tube with a top opening and a bottom opening, wherein the bottom opening is configured to be in flowable communication with a sprayable material;
an actuator coupled to a top end of the dip tube;
an expansible bladder located at the top opening of the dip tube and configured to be in flowable communication with the top opening when the actuator is depressed, wherein the expansible bladder expands in diameter from a resting diameter when the sprayable material passes through; and
a discharge opening in flowable communication with the expansible bladder, wherein sprayable material is dispensed through the discharge opening when the actuator is depressed.

2. The spray nozzle of claim 1 further including a pressure chamber located in between the expansible bladder and the discharge opening and establishing flowable communication from the expansible bladder to the discharge opening, wherein the discharge opening has a diameter that is smaller than a diameter of a portion of the expansible bladder that immediately abuts the discharge opening.

3. The spray nozzle assembly of claim 2, wherein the discharge opening has a diameter that is at least three times smaller than the diameter of the portion of the pressure chamber that immediately abuts the discharge opening.

4. The spray nozzle assembly of claim 2, wherein the pressure chamber flares to a diameter that is larger than the diameter of the dip tube.

5. The spray nozzle assembly of claim 2, wherein the pressure chamber defines a shape selected from the group consisting of bulbous, flared, square and rectangular.

6. The spray nozzle assembly of claim 1, wherein the expansible bladder comprises an elastic material.

7. The spray nozzle assembly of claim 6, wherein the elastic material is selected from the group consisting of natural rubber, synthetic rubber, silicone, and mixtures thereof.

8. The spray nozzle assembly of claim 1, wherein the expansible bladder is adapted to expand up to five times the resting diameter.

9. The spray nozzle assembly of claim 1, wherein an entry opening into the expansible bladder and an exit opening out of the expansible bladder have a diameter smaller than the resting diameter of the expansible bladder.

10. An aerosol system comprising:
a container;
a sprayable material being held in the container; and
a spray nozzle assembly attached to the container that sprays the sprayable material from the container, wherein the spray nozzle assembly further comprises
a dip tube with a top opening and a bottom opening, wherein the bottom opening is configured to be in flowable communication with the sprayable material,
an actuator coupled to a top end of the dip tube,
an expansible bladder located at the top opening of the dip tube and configured to be in flowable communication with the top opening when the actuator is depressed, wherein the expansible bladder expands in diameter from a resting diameter when the sprayable material passes through, and
a discharge opening in flowable communication with the expansible bladder, wherein sprayable material is dispensed through the discharge opening when the actuator is depressed.

11. The aerosol system of claim 10 further including a pressure chamber located in between the expansible bladder and the discharge opening and establishing flowable communication from the expansible bladder to the discharge opening, wherein the discharge opening has a diameter that is smaller than a diameter of a portion of the expansible bladder that immediately abuts the discharge opening.

12. The aerosol system of claim 11, wherein the discharge opening has a diameter that is at least three times smaller than the diameter of the portion of the pressure chamber that immediately abuts the discharge opening.

13. The aerosol system of claim 11, wherein the pressure chamber flares to a diameter that is larger than the diameter of the dip tube.

14. The aerosol system of claim 11, wherein the pressure chamber defines a shape selected from the group consisting of bulbous, flared, square and rectangular.

15. The aerosol system of claim 10, wherein the expansible bladder comprises an elastic material.

16. The aerosol system of claim 15, wherein the elastic material is selected from the group consisting of natural rubber, synthetic rubber, silicone, and mixtures thereof.

17. The aerosol system of claim 10, wherein the expansible bladder is adapted to expand up to five times the resting diameter.

18. The aerosol system of claim 10, wherein an entry opening into the expansible bladder and an exit opening out of the expansible bladder have a diameter smaller than the resting diameter of the expansible bladder.

19. A method of applying a sprayable material onto a surface area, the method comprising:
storing a sprayable material in a dispensing container having a spray nozzle assembly for application to a surface area, wherein the spray nozzle assembly further comprises
a dip tube with a top opening and a bottom opening, wherein the bottom opening is configured to be in flowable communication with the sprayable material,
an actuator coupled to a top end of the dip tube,
an expansible bladder located at the top opening of the dip tube and configured to be in flowable communication with the top opening when the actuator is depressed, wherein the expansible bladder expands in diameter from a resting diameter when the sprayable material passes through, and
a discharge opening in flowable communication with the expansible bladder, wherein sprayable material is dispensed through the discharge opening when the actuator is depressed; and
selectively applying the sprayable material onto the surface area such that a layer of the sprayable material is formed.

20. The method of claim 19 further including a pressure chamber located in between the expansible bladder and the discharge opening and establishing flowable communication from the expansible bladder to the discharge opening, wherein the discharge opening has a diameter that is smaller than a diameter of a portion of the expansible bladder that immediately abuts the discharge opening.

21. The method of claim 20, wherein the discharge opening has a diameter that is at least three times smaller than the diameter of the portion of the pressure chamber that immediately abuts the discharge opening.

22. The method of claim 20, wherein the pressure chamber flares to a diameter that is larger than the diameter of the dip tube.

23. The method of claim 20, wherein the pressure chamber defines a shape selected from the group consisting of bulbous, flared, square and rectangular.

24. The method of claim 19, wherein the expansible bladder comprises an elastic material.

25. The method of claim 24, wherein the elastic material is selected from the group consisting of natural rubber, synthetic rubber, silicone, and mixtures thereof.

26. The method of claim 19, wherein the expansible bladder is adapted to expand up to five times the resting diameter.

27. The method of claim 19, wherein an entry opening into the expansible bladder and an exit opening out of the expansible bladder have a diameter smaller than the resting diameter of the expansible bladder.
